(19) United States
(12) Patent Application Publication
(10) Pub. No.: US 2024/0286992 A1
(43) Pub. Date: Aug. 29, 2024

(54) EDDS ADDUCT COMPOSITIONS

(71) Applicant: INNOSPEC LIMITED, Ellesmere, Port Cheshire (GB)

(72) Inventor: Matthew Robert GILES, Ellesmere, Port Cheshire (GB)

(21) Appl. No.: 18/572,737

(22) PCT Filed: Jun. 13, 2022
(86) PCT No.: PCT/GB2022/051481

§ 371 Date: Dec. 20, 2023

(30) Foreign Application Priority Data
Jun. 25, 2021 (GB) 2109205.1

(51) Int. Cl. C07C 229/24; C11D 3/20; C11D 3/33; C11D 11/00

(52) U.S. Cl. CPC C07C 229/24; C11D 3/2082; C11D 3/33; C11D 11/0082

(57) ABSTRACT

An adduct of ethylenediamine disuccinic acid or a salt thereof and a further polycarboxylic acid or a salt thereof wherein the further polycarboxylic acid is selected from citric acid, polyaspartic acid, tartaric acid, malonic acid, maleic acid, carboxymethyl inulin, oxalic acid, glutaric acid, adipic acid, pimelic acid, suberic acid, azelaic acid, sebacic acid, malic acid and fumaric acid; and wherein said adduct is in the form of a free flowing particulate solid; wherein EDDS is present in the adduct in a molar excess compared with the further polycarboxylic acid.

The present invention relates to components for use in detergent compositions, to compositions comprising such components and to methods and uses relating thereto.

In particular the invention relates to components comprising builders and chelating agents.

Laundry and dishwashing detergents are used daily in significant volumes throughout the world. Much of the effluent from the use of these components finds its way into rivers and seawater. It is therefore important to minimise the levels of harmful ingredients present in detergent compositions and ensure that the components used are as effective as possible.

Automatic dishwashing and laundry compositions typically contain a mixture of surfactants, builders and bleaches with smaller amounts of enzymes and chelating agents, as well as ingredients such as foaming agents or suds suppressors, optical brighteners, fragrances, antideposition agents, corrosion inhibitors, glass protection agents and dye transfer inhibitors.

Builders are a major component of both laundry and automatic dishwashing detergents, typically making up to 50 wt % of the composition.

Builders are included as water softening agents to complex calcium ions.

Traditional builder components were phosphate based but these have been phased out due to environmental concerns.

It is highly desirable to use biodegradable builders where possible. Preferred builders include carboxylate builders.

Bleaches are also important components of automatic dishwashing and laundry detergent compositions. Typical bleaching agents included in these compositions are peroxygen based, for example hydrogen peroxide, or percarbonate or perborate salts. However transition metals present in water may cause problems in compositions containing bleaching agents as they can cause decomposition of peroxygen species. This may result in reduced bleaching performance and the creation of hydroxyl radicals which can cause fibre damage and reduced product stability. Thus it is common to add a chelating agent to compositions which include a bleaching agent, for example laundry detergent or automatic dishwashing compositions.

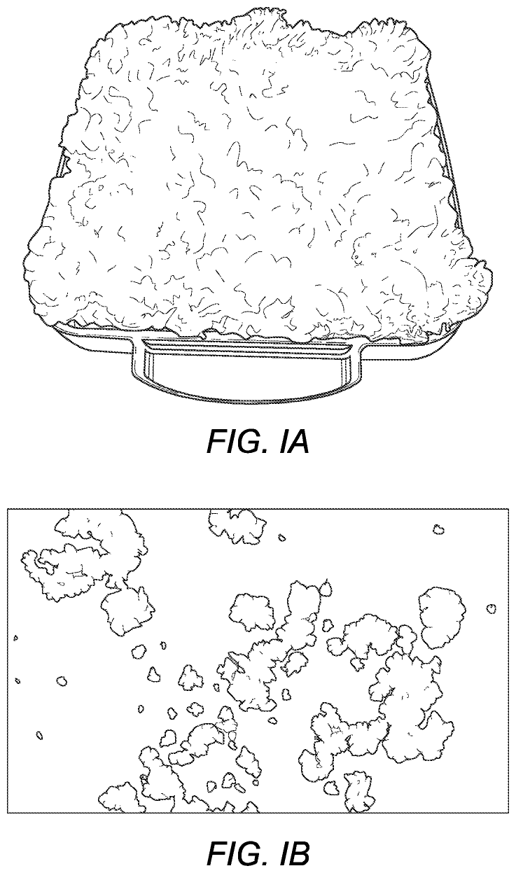

Ethylenediamine disuccinic acid (EDDS) is a commonly used chelating agent and has the structure shown in FIG. 1:

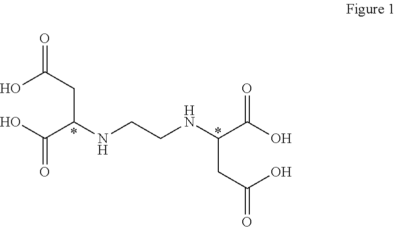 [SMILES: O=C(O)CC(NCCNC(CC(=O)O)C(=O)O)C(=O)O]

EDDS includes two stereogenic centres and there are three possible stereoisomers. A particularly preferred configuration is [S, S]-ethylenediamine disuccinic acid which is readily biodegradable.

EDDS is an effective chelating agent of transition metals and heavy metals.

EDDS is favoured over phosphonate based chelating agents due to its biodegradability.

In this specification the abbreviation “EDDS” is used to denote the structure shown in FIG. 1 and the same structure in which a number of the hydrogen atoms have been replaced. Thus EDDS may also be used to refer to succinate salts in which 1, 2, 3 or 4 of the acid groups have been neutralised or partially neutralised.

One commercially available material is trisodium ethylenediamine disuccinate. Although this compound can be prepared as a solid, the solid form is very hygroscopic and rapidly absorbs water. The commercial product is therefore supplied as an aqueous solution comprising 30% by weight EDDS (expressed as free acid), or 37 wt % of the trisodium salt (including the counterion).

Another commercially available form of EDDS is the tetra acid. This is provided as a powder which contains 65 wt % solid [S, S] EDDS as an acid and water of crystallisation.

However, each of the above mentioned commercially available materials presents problems for formulators. The tetra acid can readily be incorporated into granular compositions as it is available as a solid but has a solubility in water of just 0.3 g/Kg limiting its suitability for use in laundry and dishwashing applications. The trisodium salt may be supplied as a liquid and thus can be difficult to incorporate into solid products.

A further problem with the solid tetra acid form of EDDS is that the water of crystallisation can cause stability problems due to interactions with other components, for example bleaches, when it is incorporated into a solid formulated composition.

Many consumers are keen to use automatic dishwashing or laundry formulations in the form of a powder or compressed powder tablet but there are no commercially available products which enable readily water soluble solid forms of EDDS to be incorporated directly into laundry or dishwashing compositions.

Laundry and dishwashing detergents formulations include both chelating agents and builders.

Carboxylate based builders are favoured for their biodegradability, and EDDS is a biodegradable chelating agent.

WO2019/162136 describes compositions comprising an aminopolycarboxylate and an organic acid. The compositions are in the form of a non-crystalline solid which exhibits thermoplastic behaviour. The composition is preferably translucent and not in the form of a fine powder.

The compositions of WO2019/162136 find utility in a number of applications. However these materials cannot readily be incorporated into powdered compositions or compressed powder tablets.

The present inventors have surprisingly prepared free flowing solid compositions comprising EDDS and a further polycarboxylic acid which are readily water soluble, are not hygroscopic and are more easily incorporated into solid compositions.

According to a first aspect of the present invention there is a provided an adduct of ethylenediamine disuccinic acid (EDDS) or a salt thereof and a further polycarboxylic acid or a salt thereof wherein the further polycarboxylic acid is selected from citric acid, polyaspartic acid, tartaric acid, malonic acid, maleic acid, carboxymethyl inulin, oxalic acid, glutaric acid, adipic acid, pimelic acid, suberic acid, azelaic acid, sebacic acid, malic acid and fumaric acid; wherein said adduct is in the form of a free flowing particulate solid; and wherein EDDS is present in the adduct in a molar excess compared with the further polycarboxylic acid.

Preferably the further polycarboxylic acid is selected from citric acid, polyaspartic acid, tartaric acid, malonic acid, maleic acid and carboxymethyl inulin.

Ethylene diamine disuccinic acid has the structure shown in FIG. 1 above.

Citric acid has the structure shown in FIG. 2:

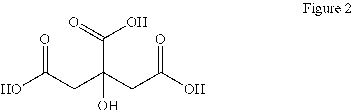 [SMILES: O=C(O)CC(O)(CC(=O)O)C(=O)O]

In this specification citric acid may be used to refer to the structure shown in FIG. 2 or the same structure in which a number of the hydrogen atoms have been replaced. Thus citric acid may also be used to refer to citric acid salts in which 1, 2, or 3 of the acid groups have been neutralised or partially neutralised.

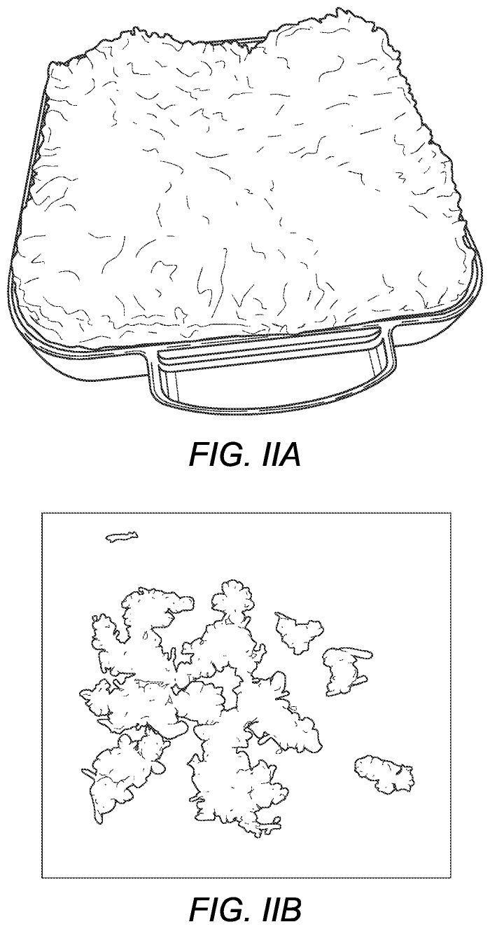

Polyaspartic acid is a polymer of aspartic acid, HOOCCH₂CHNH₂COOH. It is formed by amide bonds between the amine group and either acid group. The polymer thus includes repeating units A and B shown in FIG. 3.

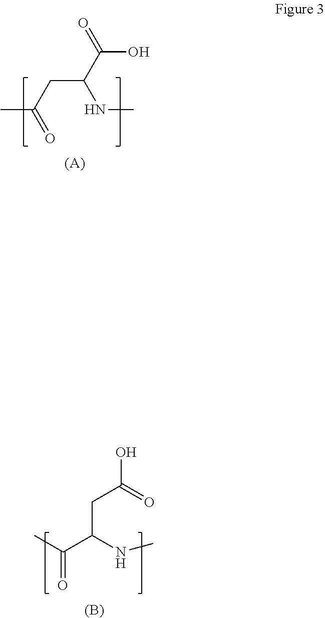

Monomer units (A) and (B) may be randomly present in any order throughout the polymer structure. Each monomer unit may have either stereochemistry.

In this specification, polyaspartic acid may refer to a polymer of the units shown in FIG. 3 or the same structure in which a number of the hydrogen ions have been replaced.

One preferred source of polyaspartic acid is sold under the trade mark Baypure DS100. This material has the structure shown in FIG. 3(C):

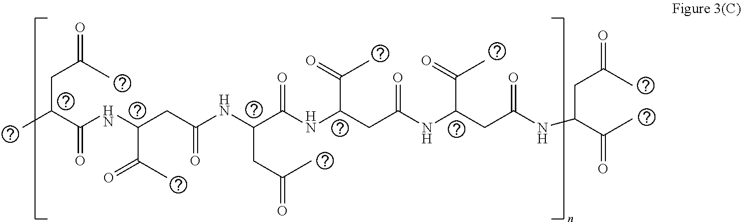

Polyaspartic acids suitable for use in the present invention preferably have a number average molecular weight of at least 500, preferably at least 1000. The number average molecular weight may be up to 60000, suitably up to 50000, preferably up to 25000, for example up to 10000. Preferred polyaspartic acids for the use herein have a number average molecular weight of 1000 to 5000, suitably 1500 to 4000, for example 2000 to 3000.

Tartaric acid has the structure shown in FIG. 4:

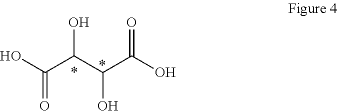

Tartaric acid includes two stereogenic centres and there are three possible stereoisomers. A particularly preferred configuration is [S,S]-tartaric acid.

In this specification tartaric acid may refer to the structure shown in FIG. 4 or the same structure in which a number of the hydrogen atoms have been replaced.

Malonic acid has the structure shown in FIG. 5:

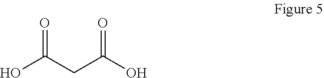

In this specification malonic acid may refer to the structure shown in FIG. 5 or the same structure in which a number of the hydrogen atoms have been replaced.

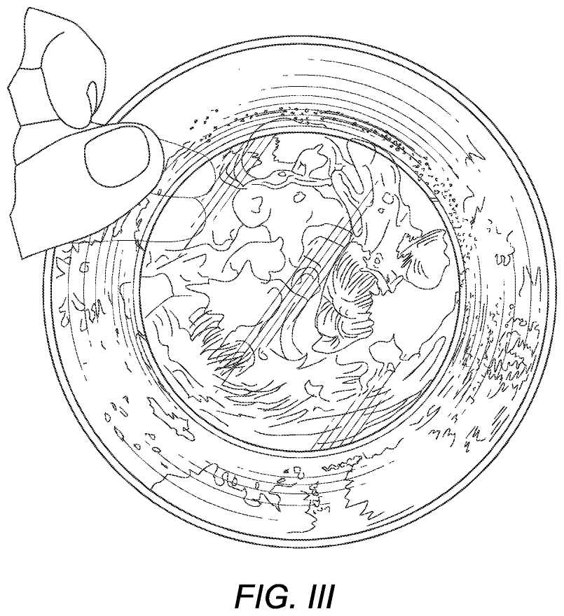

Maleic acid has the structure shown in FIG. 6:

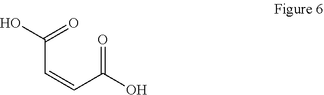

In this specification maleic acid may refer to the structure shown in FIG. 6 or the same structure in which a number of the hydrogen atoms have been replaced.

Carboxymethyl inulin has the structure shown in FIG. 7:

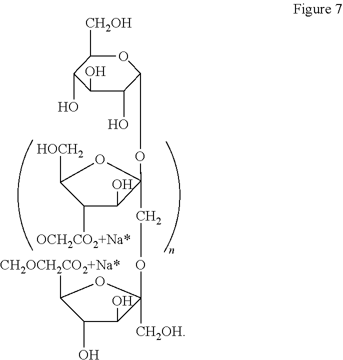

In this specification carboxymethyl inulin may refer to the structure shown in FIG. 7 or the same structure in which a number of the hydrogen atoms have been replaced.

With reference to FIGS. 1 to 7 which cover embodiments in which one or more of the hydrogen atoms may be replaced, these are suitably replaced by a cation to provide a salt of one or more of the acid moieties present in the molecule.

Suitably one or more hydrogen atoms may be replaced by an ammonium ion, a substituted ammonium ion, an alkali metal ion, an alkaline earth metal or a transition metal ion.

Preferably they are replaced by an alkali metal ion, for example sodium or potassium, preferably a sodium ion.

The adduct of the present invention is formed from ethylenediamine disuccinic acid or a salt thereof and a further polycarboxylic acid or a salt thereof. In some embodiments the adduct may comprise a mixture of two or more further polycarboxylic acids. As such references to a further polycarboxylic acid or further polycarboxylic acid cover embodiments in which the adduct comprises two or more further polycarboxylic acids.

Suitably the further polycarboxylic acid is selected from polyaspartic acid and citric acid.

Most preferably the further polycarboxylic acid is citric acid.

Thus in especially preferred embodiments the present invention provides an adduct of ethylenediamine disuccinic acid or a salt thereof and citric acid wherein said adduct is in the form of a free flowing particulate solid.

The adduct of the present invention comprises a number of carboxylic acid groups. By carboxylic acid groups we mean to refer to the free acid moieties COOH and the moieties COOM where M is an ammonium ion or a metal ion, preferably an alkali metal ion, especially sodium.

The adduct of the present invention comprises a molar excess of EDDS compared to the further polycarboxylic acid. By this we mean that the number of moles of EDDS present in the adduct is greater than the number moles of the further carboxylic acid.

Each mole of EDDS provides 4 moles of carboxylic acid groups to the adduct.

The number of carboxylic acid groups provided by the further polycarboxylic acid depends on the nature of the further polycarboxylic acid. For example each mole of citric acid provides 3 carboxylic acid groups to adduct.

Preferably from 60 to 95 mol % of the carboxylic acid groups present in the adduct are provided by ethylenediamine disuccinic acid and 40 to 5 mol % of the carboxylic acid groups present in the adduct are provided by the further polycarboxylic acid.

Preferably at least 65 mol % of the carboxylic acid groups present in the adduct are provided by EDDS.

In some preferred embodiments in which the further polycarboxylic acid is citric acid, preferably from 65 to 95 mol % of the carboxylic acid groups present in the adduct are provided by ethylenediamine disuccinic acid and 35 to 5 mol % of the carboxylic acid groups present in the adduct are provided by citric acid.

In some preferred embodiments the molar ratio of EDDS to citric acid is preferably from 55:45 to 95:5, for example from 60:40 to 90:10 or from 65:35 to 85:15.

The adduct of the present invention suitably consists essentially of EDDS and the further polycarboxylic acid.

The adduct of the present invention is a distinct chemical/physical entity and not simply a mixture of its starting materials.

The adduct of the present invention is in the form of a solid. By this we mean the material is a solid at atmospheric temperature and pressure (20 to 25° C., 1 atmosphere).

The adduct of the present invention is in the form of a free flowing particulate material.

Preferably the adduct of the present invention is not amorphous and is in crystalline or semi-crystalline form.

Preferably the adduct may be easily incorporated into a granular composition.

Preferably the adduct of the present invention is not hygroscopic under normal atmospheric conditions. Preferably it absorbs less than 10% of its weight of water upon exposure to standard atmospheric conditions for 7 days, preferably less than 5% of its weight.

The adduct of the present invention is preferably water soluble. In the concentrations at which it is typically included in, for example, laundry and dishwashing applications, it fully dissolves quickly.

According to a second aspect of the present invention there is provided a method of preparing an adduct of ethylenediamine disuccinic acid or a salt thereof and a further polycarboxylic acid or a salt thereof wherein the further polycarboxylic acid is selected from citric acid, polyaspartic acid, tartaric acid, malonic acid, maleic acid, carboxymethyl inulin, oxalic acid, glutaric acid, adipic acid, pimelic acid, suberic acid, azelaic acid, sebacic acid, malic acid and fumaric acid, the method comprising:
(i) admixing a composition comprising ethylenediamine disuccinic acid or a salt thereof with a composition comprising the further polycarboxylic acid or a salt thereof wherein EDDS is provided in a molar excess compared with the further carboxylic acid; and
(ii) collecting the adduct in the form of a free flowing particulate solid.

Further features of the first and second aspect will now be described. The method may optionally include one or more further steps between step (i) and step (ii).

Preferred features of the second aspect are as defined in relation to the first aspect.

The composition comprising EDDS used in step (i) may include EDDS in any suitable form. Preferably EDDS is provided in a form which is water soluble. Preferably the EDDS is present as a metal salt, preferably as a salt of an alkali metal or alkaline earth metal. Most preferably it is present as a sodium salt.

Preferably the EDDS is present in the form of a sodium salt comprising from 1 to 4 moles of sodium, more preferably from 2.5 to 3.5 moles of sodium per mole of EDDS.

Most preferably EDDS is present as a compound having the empirical formula of trisodium ethylenediamine disuccinate.

The adduct of the first aspect of the present invention is preferably the adduct of a water soluble salt of EDDS and a further polycarboxylic acid selected from citric acid, polyaspartic acid, tartaric acid, malonic acid, maleic acid and carboxymethyl inulin. The exact structure of the adduct is not known but it is thought to be a salt or other coordination complex.

The adduct of the first aspect of the present invention is preferably the adduct of trisodium EDDS and a further polycarboxylic acid selected from citric acid, polyaspartic acid, tartaric acid, malonic acid, maleic acid and carboxymethyl inulin. The exact structure of the adduct is not known but it is thought to be a salt or other coordination complex.

Most preferably the adduct of the first aspect of the present invention is preferably the adduct of trisodium EDDS and citric acid.

Citric acid may be provided in the form of a solid powder as the free acid or a sodium salt of the acid. Citric acid may be provided as a solution. Preferably citric acid is provided as the free acid. The EDDS portion of the adduct of the present invention may include any of the steroisomers. Thus it may be selected from [R,R]-EDDS, [R,S]-EDDS, [S, S]-EDDS and any combination thereof.

Preferably the EDDS is present in substantially the [S,S]-form. Preferably at least 70%, more preferably at least 90% of the EDDS is of the [S,S] configuration.

Preferably the composition comprising EDDS is an aqueous solution of EDDS.

When the further polycarboxylic acid comprises tartaric acid this may be present as the [R,R], [S,S] or [R,S] isomer. Preferably tartaric acid is present in substantially the [S,S]-form. Preferably at least 70%, more preferably at least 90% of the tartaric acid is of the [S, S] configuration.

In preferred embodiments the further polycarboxylic acid is selected from polyaspartic acid and citric acid.

In especially preferred embodiments the further polycarboxylic acid is citric acid.

Preferably the composition comprising the further polycarboxylic acid is an aqueous solution of the further polycarboxylic acid.

Preferably the method of the second aspect involves mixing a solution, preferably an aqueous solution, of a salt of EDDS with a solution, preferably an aqueous solution, of the further polycarboxylic acid or a salt thereof.

Preferably the solution of EDDS comprises a sodium salt of EDDS, more preferably trisodium EDDS.

Suitably the salt of EDDS is provided as an aqueous solution comprising from 20 to 50 wt %, more preferably 25 to 35 wt % EDDS (expressed as EDDS free acid), and preferably the further polycarboxylic acid is provided as an aqueous solution comprising from 20 to 80 wt %, more preferably 30 to 60 wt % active (expressed as equivalent free acid).

In some preferred embodiments no further water is added to the reaction mixture in the method of the second aspect.

Step (i) of the method of the second aspect involves admixing a composition comprising EDDS or a salt thereof with a composition comprising the further polycarboxylic acid or a salt thereof.

The method may optionally include a further step of adjusting the pH of the mixture provided in step (i).

Suitably the pH is adjusted to provide a mixture having a pH of at least 4.5, preferably at least 5, for example at least 5.5.

Suitably the pH is adjusted to provide a mixture having a pH of less than 10, preferably less than 9, for example less than 8.5.

Suitably step (i) of the method of the second aspect involves admixing a composition comprising EDDS or a salt thereof with a composition comprising the further polycarboxylic acid or a salt thereof, and optionally adjusting the pH to provide a mixture having a pH of from 5 to 9, preferably from 5.5 to 8.5.

When the further polycarboxylic acid is citric acid, preferably the pH is not adjusted.

In some embodiments admixture of the composition comprising EDDS or a salt thereof and the composition comprising the further polycarboxylic acid or a salt thereof may directly provide a composition having a pH of between 5 and 9. This will depend on the nature of the further polycarboxylic acid and whether this component and the EDDS component is provided as the free acid or a salt thereof. In some embodiments the pH may need to be adjusted to provide a composition having a pH of between 5 and 9.

Any suitable reagents may be used to adjust the pH. Such reagents will be known to the person skilled in the art. Preferred reagents include sodium hydroxide and hydrochloric acid.

The resulting mixture may optionally be agitated. Suitably precipitation of the adduct follows.

Step (ii) involves collecting the adduct in the form of a free flowing particulate solid.

Precipitation of the adduct may occur following admixture and optional agitation. In some embodiments crystallisation may be induced.

In step (ii) of the method of the second aspect residual water may be removed using methods known to those skilled in the art, for example by filtration, evaporation, or heating under reduced pressure.

In some preferred embodiments water is simply allowed to evaporate from the composition obtained in step (i) leading to precipitation of the adduct.

The method of the second aspect of the invention may involve heating the mixture obtained in step (i). Preferably the mixture is not heated above 50° C. More preferably the mixture obtained in step (i) is not heated above 40° C. Preferably the mixture directly obtained in step (i) is not heated.

The solid particulate material obtained in step (ii) may be gently heated, preferably to a temperature of less than 50° C. to assist drying.

In some embodiments the composition may be spray dried. Spray granulation may also be used.

According to a third aspect of the present invention, there is provided a composition comprising an adduct of the first aspect.

The composition may consist essentially of the adduct of the first aspect or it may include one or more further components. The composition of the second aspect may be a solid composition or a liquid composition. It may be a composition of any type in which EDDS and/or the further polycarboxylic acid has previously been used. In preferred embodiments the composition is a laundry detergent composition or an automatic dishwashing composition.

In some embodiments the composition may be in the form of a particulate material, for example a free flowing powder. In some embodiments the composition may be in the form of compressed tablets In some embodiments the composition may be provided as a unit dose in liquid or solid form, in the shell of water-soluble polymeric material.

The composition may be a granular composition.

Solid laundry compositions of the present invention preferably comprise from 0.01 to 20 wt %, more preferably 0.01 to 4 wt %, most preferably 0.1 to 1 wt % of an adduct of the first aspect.

Liquid laundry compositions of the present invention preferably comprise from 0.01 to 50 wt %, more preferably 0.1 to 20 wt %, most preferably 1 to 10 wt % of an adduct of the first aspect.

Automatic dishwashing compositions of the present invention preferably comprise 0.1 to 75 wt % of an adduct of the first aspect, more preferably 1 to 50 wt % and most preferably 2 to 25 wt %.

In some embodiments the composition of the third aspect may comprise a mixture of two or more adducts of the first aspect. In such embodiments the above amounts refer to the total amount of all such adducts present in the composition.

Adducts of the present invention find utility in solid formulations as they may be directly incorporated without removal of water. They are also useful for formulators of liquid compositions as solids are often easily to handle and dose accurately and there is no concurrent addition of water.

Laundry and dishwashing compositions of the present invention preferably comprise further ingredients selected from surfactants, builders, bleaches, bleach activators, redeposition additives, dye transfer inhibitors, enzymes, colorants and fragrances.

The present invention may further provide a method of manufacturing a detergent composition, preferably an automatic dishwashing or laundry composition, the method comprising:
admixing an aqueous solution of EDDS or a salt thereof and an aqueous solution of a further polycarboxylic acid or a salt thereof wherein EDDS is provided in a molar excess compared with the further polycarboxylic acid;
optionally adjusting the pH of the resultant mixture;
   collecting the precipitate that forms; and
   admixing the precipitate with one or more further components.

The provision of a single combined source of a further polycarboxylic acid, preferably citric acid and EDDS in solid form provides a considerable improvement in the formulation possibilities for manufacturers of products containing these materials. This is particularly valuable in the case of laundry and dishwashing compositions. However, other compositions are also within the scope of the present invention. For example, the composition of the second aspect may comprise a bleaching composition, a cleaning composition, an agricultural composition or a personal care composition.

The present invention further provides the use of an adduct of the first aspect in one of the ways in which known commercial sources of EDDS and/or the further polycarboxylic acid have previously been used.

The present invention provides the use of an adduct of the first aspect as a chelating agent. It may be used as a chelating agent for binding transition metals or alkaline earth metals. Preferably the adduct of the first aspect is used as a chelating agent in environments in which transition metals and alkaline earth metals, especially calcium, are found.

The present invention includes the use of an adduct of the first aspect in detergent compositions, for example laundry or automatic dishwashing compositions.

The present invention also includes the use of an adduct of the first aspect in other applications, for example agricultural applications (e.g. slug pellets, herbicides, foliar feeds, nutrient feeds, hydroponics); pulp and paper bleaching (including mechanical bleaching, chemical bleaching, thermochemical bleaching, during both the Q-stage and the P-stage); personal care applications (hair care, soap bars, syndet bars, creams); cleaning applications (household, institutional and industrial); oil field applications (scale remover); metal cleaning applications (PCB, electroless plating); as a biocide potentiator; in medical applications (anti-poison, metal delivery); and in food applications, for example as a stabiliser or antioxidant.

The adduct of the first aspect also finds considerable utility as an anti scalant agent.

Thus the present invention includes the use of an adduct of the first aspect in detergent compositions, for example laundry or automatic dishwashing compositions.

The present invention includes the use of a salt of the first aspect in agricultural applications. For example the salt may be used in slug pellets, in herbicides, in foliar feeds, in nutrient feeds and in hydroponics.

The present invention provides the use of an adduct of the first aspect in pulp and paper bleaching. This includes mechanical bleaching and chemical bleaching as well as thermo-mechanical bleaching. The salt of the first aspect may be used in the Q stage and the P stage of the pulp bleaching, that is the wash in which metals are removed and the peroxide stage in which bleaching occurs. These terms are well understood to those skilled in the art.

The present invention provides the use of an adduct of the first aspect in personal care applications. For example the salt may be incorporated in hair care compositions, for example hair dyes and shampoo. It may also be included as an antioxidant in creams, for example sun creams and the like. It may be included in dental compositions, for example toothpaste or mouthwashes.

The present invention includes the use of an adduct of the first aspect as a biocide potentiator. As such it may be able to increase the effectiveness of a biocide and may find wide application. For example it may be used in personal care applications.

The present invention may provide the use of an adduct of the first aspect in household, institutional and industrial cleaning applications. It may be included in hard surface cleaners, bathroom and kitchen cleaners, in bottle washing applications or in the cleaning of dairy equipment.

The present invention may provide the use of an adduct of the first aspect as an anti-scalant material, for example as a sequestrant of calcium and magnesium salts.

The adduct of the present invention may be used in oil field applications as a scale remover, for example to remove barium and strontium salts.

The present invention may provide the use of an adduct of the first aspect in metal cleaning applications, for example printed circuit boards or electroless plating applications.

The present invention may provide the use of an adduct of the first aspect in medical applications, for example, as an anti-poison material. The present invention may be used to assist the delivery of metals to parts of the body.

The present invention will now be further described by way of the following non-limiting examples.

### EXAMPLES 1 TO 14

The adducts detailed in table 1 were prepared according to the following method.

The appropriate amount of a 37 wt % aqueous solution of trisodium EDDS was mixed with the stated amount of a concentrated aqueous solution of the further carboxylic acid (or salt as specified), with stirring.

Residual water was allowed to evaporate. Examples 1 to 3 and 5 to and 12 are of the invention. Examples 4, 13 and 14 are comparative.

In table 1 the ratio EDDS:other is the molar ratio of the EDDS component to the other acid component present in the adduct. The reference to mol % COOX refers to the percentage of acid residues provided by that component as a proportion of the total number of acid residues COOX present in the adduct from both components.

TABLE 1
Preparation of adducts

| | | EDDS:other | mol % | mol % | | |
|---|---|---|---|---|---|---|
| | Polycarboxylic | (molar ratio | COOX | COOX | pH of | |
| Example | acid | as acid) | from EDDS | from other | mixture | Form of product |
| 1 | Citric acid | 90:10 | 92.3 | 7.7 | 7 | White solid, non |
| | | | | | (adjusted)* | hygroscopic |
| 2 | Citric acid | 75:25 | 80.0 | 20.0 | 7.4 | White solid, non |
| | | | | | | hygroscopic |
| 3 | Citric acid | 60:40 | 66.7 | 33.3 | 5.2 | White solid, non |
| | | | | | | hygroscopic |
| 4 (comp) | Citric acid | 50:50 | 57.1 | 42.9 | 4.6 | Sticky solid, hygroscopic |
| 5 | Sodium | 75:25 | 92.3 | 7.7 | 7 | Amber solid, non |
| | polyaspartate | | | | (adjusted)* | hygroscopic |
| 6 | Sodium | 60:40 | 85.7 | 14.3 | 7.1 | Amber solid, non |
| | polyaspartate | | | | (adjusted)* | hygroscopic |
| 7 | Tartaric acid | 75:25 | 85.7 | 14.3 | 8.1 | White solid, non |
| | | | | | | hygroscopic |
| 8 | Tartaric acid | 60:40 | 75.0 | 25.0 | 5.8 | White solid, non |
| | | | | | | hygroscopic |
| 9 | Malonic acid | 60:40 | 75.0 | 25.0 | 6.8 | White solid, non |
| | | | | | | hygroscopic |
| 10 | Maleic acid | 75:25 | 85.7 | 14.3 | 8.0 | White solid, non |
| | | | | | | hygroscopic |
| 11 | Maleic acid | 60:40 | 75.0 | 25.0 | 6.6 | White solid, non |
| | | | | | | hygroscopic |
| 12 | Carboxymethyl | 60:40 | 85.7 | 14.3 | 6.5 | Yellow solid, non |
| | inulin | | | | (adjusted)* | hygroscopic |
| 13 (comp) | Succinic acid | 60:40 | 75.0 | 25.0 | 7.6 | Glassy solid |
| 14 (comp) | Lactic acid | 60:40 | 85.7 | 14.3 | 8.6 | Hard hygroscopic solid |

*The pH was suitably adjusted by the addition of NaOH or HCl as appropriate.

Figures IA and IB show the material of example 2.

Figures IIA and IIB show the material of example 3.

### EXAMPLE 15 (COMPARATIVE)

The material of example 4 (table A) of WO2019/162136 was prepared following the procedure set out on page 30 of that document. The material obtained was a hard glassy solid and is shown in FIG. 111.

1. An adduct of ethylenediamine disuccinic acid or a salt thereof and a further polycarboxylic acid or a salt thereof wherein the further polycarboxylic acid is selected from citric acid, polyaspartic acid, tartaric acid, malonic acid, maleic acid, carboxymethyl inulin, oxalic acid, glutaric acid, adipic acid, pimelic acid, suberic acid, azelaic acid, sebacic acid, malic acid and fumaric acid; and wherein said adduct is in the form of a free flowing particulate solid; wherein EDDS is present in the adduct in a molar excess compared with the further polycarboxylic acid.

2. The adduct according to claim 1 wherein the further polycarboxylic acid is selected from citric acid, polyaspartic acid, tartaric acid, malonic acid, maleic acid and carboxymethyl inulin, wherein said adduct is in the form of a solid.

3. The adduct according to claim 1, wherein from 60 to 95 mol % of the carboxylic acid groups present in the adduct are provided by ethylenediamine disuccinic acid and 40 to 5 mol % of the carboxylic acid groups present in the adduct are provided by the further polycarboxylic acid.

4. A method of preparing an adduct of ethylenediamine disuccinic acid or a salt thereof and a further polycarboxylic acid or a salt thereof wherein the further polycarboxylic acid is selected from citric acid, polyaspartic acid, tartaric acid, malonic acid, maleic acid, carboxymethyl inulin, oxalic acid, glutaric acid, adipic acid, pimelic acid, suberic acid, azelaic acid, sebacic acid, malic acid and fumaric acid, the method comprising:
(i) admixing a composition comprising ethylenediamine disuccinic acid or a salt thereof with a composition comprising the further polycarboxylic acid or a salt thereof wherein EDDS is provided in a molar excess compared with the further polycarboxylic acid; and
(ii) collecting the adduct in the form of a free flowing particulate solid.

5. The adduct of claim 1, wherein the source of ethylenediamine disuccinic acid is a sodium salt thereof.

6. The adduct according to claim 5 wherein the source of ethylenediamine disuccinic acid is trisodium ethylenediamine disuccinate.

7. The adduct of claim 1, wherein the further polycarboxylic acid is citric acid.

8. The adduct of claim 1, wherein the adduct is not hygroscopic.

9. The adduct of claim 1, wherein the adduct is water soluble.

10. The method of preparing an adduct as claimed in claim 4 which method involves mixing an aqueous solution of a salt of EDDS with an aqueous solution of the further polycarboxylic acid or a salt thereof.

11. The method according to claim 10 which includes a step of adjusting the pH to provide a mixture having a pH of between 4.5 and 10.

12. The method according to claim 4 wherein the adduct precipitates from the mixture obtained in step (i).

13. A composition comprising an adduct of claim 1.

14. The composition as claimed in claim 13 which is a granular composition.

15. The composition as claimed in claim 13 which is a laundry detergent or automatic dishwashing composition.

16. A method of manufacturing a detergent composition, the method comprising:
admixing an aqueous solution of EDDS or a salt thereof and an aqueous solution of a further polycarboxylic acid or a salt thereof wherein EDDS is provided in a molar excess compared with the further polycarboxylic acid;
optionally adjusting the pH of the resultant mixture;
collecting the precipitate that forms; and
admixing the precipitate with one or more further components.

17. The use of an adduct as defined in claim 1 as a chelating agent.

18. The use of claim 16 in an environment in which transition metals and alkaline earth metals are present.

19. The use of an adduct as defined in claim 1 as an antiscalant.

20. The use of an adduct as claimed in claim 1 in agricultural applications (e.g. slug pellets, herbicides, foliar feeds, nutrient feeds, hydroponics); pulp and paper bleaching (including mechanical bleaching, chemical bleaching, thermochemical bleaching, during both the Q-stage and the P-stage); personal care applications (hair care, soap bars, syndet bars, creams); cleaning applications (household, institutional and industrial); oil field applications (scale remover); metal cleaning applications (PCB, electroless plating); as a biocide potentiator; in medical applications (anti-poison, metal delivery); and in food applications, for example as a stabiliser or antioxidant.

21. The use of an adduct as claimed in claim 1, in any of agricultural applications, bleaching applications, cleaning applications, medical applications or personal care applications.
